(19) United States
(12) Patent Application Publication
(10) Pub. No.: US 2007/0078363 A1
(43) Pub. Date: Apr. 5, 2007

(54) Limb protection apparatus

(76) Inventors: Edwin Kuehnle, Doylestown, PA (US); Thomas Maschi, New Hope, PA (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/401,110

(22) Filed: Apr. 10, 2006

Related U.S. Application Data
(63) Continuation-in-part of application No. 11/243,092, filed on Oct. 4, 2005.

(51) Int. Cl. A61F 5/00

(52) U.S. Cl. 602/12; 602/26; 602/5

(57) ABSTRACT

The present invention is a limb protection apparatus having a torsion spring assembly. Further, the present invention is a limb protection apparatus having a torsion spring assembly having a preload. The spring assembly has a spring and two rotatably coupled components disposed within the spring, wherein the two components cannot rotate a full 360 degrees in relation to each other, thereby allowing the assembly to be placed in a preload state.

### CROSS REFERENCE TO RELATED APPLICATIONS

This application is a continuation-in-part of U.S. patent application Ser. No. 11/243,092, filed Oct. 4, 2005, the contents of which are incorporated herein by reference in their entirety.

### FIELD OF THE INVENTION

The present invention relates to a limb protection apparatus having a torsion spring assembly. Further, the present invention relates to a limb protection apparatus having a torsion spring assembly having a preload.

### BACKGROUND OF THE INVENTION

Torsion springs, which are used in many devices, including knee braces, hinges, etc., are designed to be activated rotationally and provide an angular return force. A typical torsion spring has no preload. That is, the typical torsion spring does not have an angular return force being applied when the spring is at rest or not being used. Those torsion springs that do have a preload require an adjustment mechanism in order to create the preload. There is a need in the art for a torsion spring having a preload.

### BRIEF SUMMARY OF THE INVENTION

The present invention, in one embodiment, is a spring assembly having a first substantially cylindrical component, a second substantially cylindrical component coupled with the first substantially cylindrical component, a coupling component configured to prevent full 360 degree rotation of the first and second substantially cylindrical components in relation to each other, and a spring encircling at least a portion of the first and second components. The spring has a preload created by the first and second component and is operably coupled to a limb protection apparatus.

The present invention, in another embodiment, is a limb protection apparatus having a spring assembly and a limb guard component. The spring assembly has a first substantially cylindrical component, a second substantially cylindrical component coupled with the first component, and a coupling component configured to prevent full 360 degree rotation of the first and second substantially cylindrical components in relation to each other. Further spring assembly has a spring encircling at least a portion of the first and second components, a first connection component associated with a second end of the first substantially cylindrical component, and a second connection component associated with a second end of the second substantially cylindrical component. The spring has a pre-load tension created by the first and second components. The limb guard is operably coupled with the first and second connection components.

In another embodiment, the present invention is a limb protection apparatus having a torsion spring assembly having a preload, a first member, a second member, and a limb guard component operably coupled to the first and second members. The spring assembly has a first substantially cylindrical component, a second substantially cylindrical component, and a spring disposed substantially around the first and second substantially cylindrical components. The first substantially cylindrical component has a first connection component and a first coupling component and the second substantially cylindrical component has a second connection component and a second coupling component. The second coupling component is rotatably coupled with the first coupling component, whereby the first substantially cylindrical component rotates less than 360 degrees. The spring is connected at a first end to the first substantially cylindrical component and at a second end to the second substantially cylindrical component. The first member is operably coupled to the first connection component and the second member is operably coupled to the second connection component.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a perspective view of a spring assembly, according to one embodiment of the present invention.

FIG. 2 is a perspective view of a spring and two cylindrical components, according to one embodiment of the present invention.

FIG. 3 is a perspective view of two coupled cylindrical components, according to one embodiment of the present invention.

FIG. 4 is a perspective view of a spring assembly, according to another embodiment of the present invention.

FIG. 5 is a schematic drawing of a spring assembly, according to a further embodiment of the present invention.

FIG. 6 is a schematic drawing of a spring assembly coupled with a limb protection apparatus, according to one embodiment of the present invention.

### DETAILED DESCRIPTION

The present invention relates to a limb protection apparatus having a torsion spring assembly, including a torsion spring assembly with a preload. The assembly of the present invention can be used with known limb protection devices such as leg guards, shin guards, and the like. According to one embodiment, the present invention provides both limb protection and benefits provided by limb brace devices.

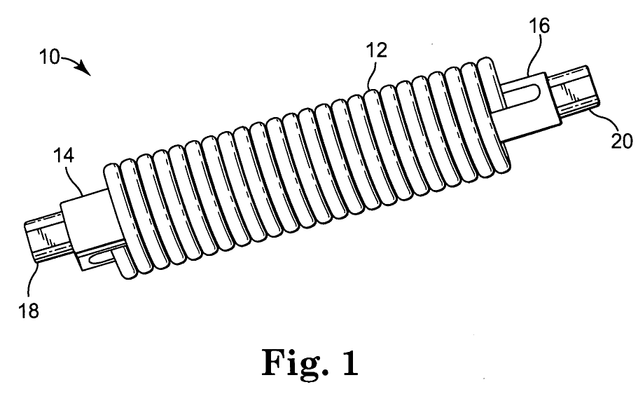

FIG. 1 illustrates a torsion spring assembly 10, according to one embodiment of the present invention. The assembly 10 has a coiled spring 12 encircling two cylindrical components 14, 16. Each cylindrical component 14, 16 has a connection component 18, 20 configured to engage with any device or component that is to be placed under rotational tension with respect to another different component or device attached at the opposite connection component.

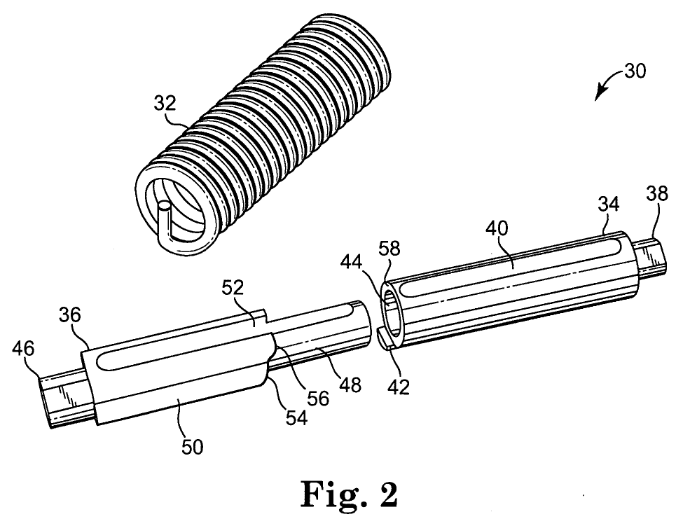

FIG. 2 depicts a disassembled torsion spring assembly 30, according to a further aspect of the present invention. The assembly 30 has a spring 32 and two cylindrical components 34, 36. The cylindrical component 34 is also referred to herein as a “female component” and the cylindrical component 36 is also referred to herein as a “male component.” The female component 34, in accordance with one embodiment, has a connection component 38, a spring receiving channel 40, a protrusion 42 or “tang”, and an opening 44 in fluid communication with a hollow portion (not shown) in the component 38.

The male component 36, according to one embodiment, has a connection component 46, a first or “small” portion 48, a second or “large” portion 50, and a spring receiving channel 52. The first portion 48 has a diameter that is smaller than the second portion 50 such that the male component 36 has a shoulder 54. The shoulder 54 has a protrusion 56 or extending toward the small portion 48.

When assembled, the two cylindrical components 34, 36 according to one embodiment are coupled together such that the small portion 48 of the male component 36 is inserted into the opening 44 of the female component 34 and the tang 42 of the female component 34 contacts the shoulder 56 of the male component 36. The spring 32 is positioned such that the spring 32 encircles at least a portion of the two cylindrical components 34, 36 and one end of the spring 32 is inserted into the spring receiving channel 40 and the other end of the spring 32 is inserted into the spring receiving channel 52, as best shown in the embodiment depicted in FIG. 1.

The connection components 38, 46 depicted in FIG. 2 are “D-shaped” protrusions 38, 46, according to one embodiment of the present invention. That is, each protrusion 38, 46 has a substantially circular profile that includes a flat portion such that the profile of the protrusion 38, 46 looks somewhat like a “D”. The protrusions 38, 46 are configured to connect or “mate” snugly with corresponding holes in the components (not shown) intended to be connected to the spring assembly 30. Alternatively, the connection components 38, 46 are any known components for connection to devices or components (not shown) of a device intended to be connected to the spring assembly 30.

Each spring receiving channel 40, 52 is configured to receive one end of the spring 32. According to one embodiment as depicted, each channel 40, 52 is a channel-shaped opening that is positioned longitudinally on the side of each component 34, 36 and is almost as long as each component 34, 36. This configuration allows for easy assembly and disassembly of the apparatus. Alternatively, each channel 40, 52 can be any opening of any shape that allows for receiving and retaining an end of the spring 32.

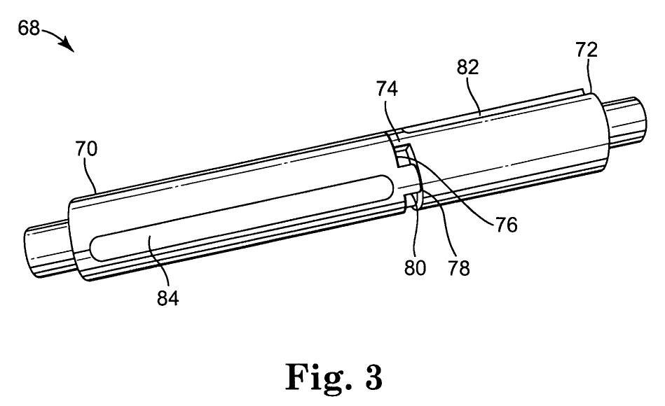

FIG. 3 depicts the operable coupling of female 70 and male 72 components of an assembly 68 in which no spring is shown. The components 70, 72 are coupled such that the protrusion 74 on the male component 72 is in contact with the end 76 of the female component 70 and the tang 78 of the female component 76 is in contact with the shoulder 80 of the male component 72. In this configuration, the components 70, 72 can be rotated in relation to each other, but only until the tang 78 and the protrusion 74, which are positioned along substantially the same axis, come into contact with each other. That is, the tang 78 and the protrusion 74 prevent the components 70, 72 from rotating a full 360° in relation to each other. As either component 70, 72 is rotated in relation to the other, the tang 78 and the protrusion 74 eventually come into contact, preventing further rotation.

The configuration of the spring assembly 68 as depicted in FIGS. 2 and 3 creates a preload, in accordance with one embodiment of the present invention. “Preload” is spring-created tension that exists while the spring is not in use or prior to use. In one aspect of the invention, the coupling of the two components 70, 72 as discussed above provides the preload. That is, to provide a preload, the assembly 68 is configured such that when a spring is added to the assembly 68 as shown in the embodiment depicted in FIG. 1, one end of the spring is inserted into the spring receiving channel 82 and the other is inserted into the spring receiving channel 84 such that a tension is created upon insertion of both ends. The tension causes the components 70, 72 to rotate in relation to one another until the tang 78 and the protrusion 74 come into contact, thereby preventing further rotation and preventing the spring from releasing the tension. Thus, the tension is maintained as preload. In an alternative embodiment, any known coupling that prevents full 360 degree rotation of the male and female components in relation to one another and thereby creates a preload when operably coupled to a spring can be implemented into the spring assembly of the present invention.

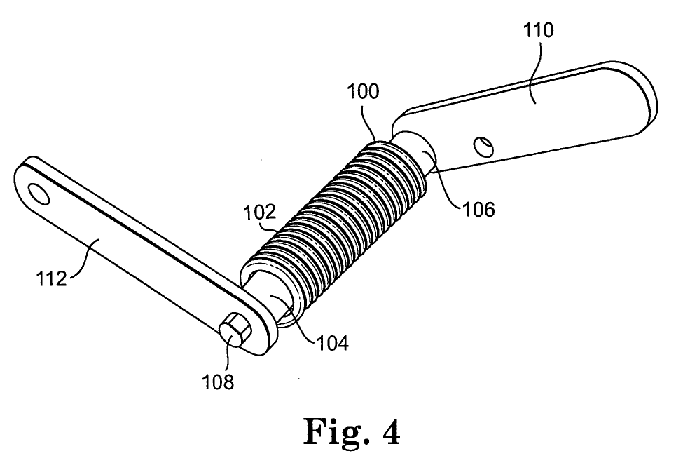

FIG. 4 depicts a spring assembly 100 according to one embodiment that is operably coupled to components 110, 112 to be placed under torsion tension. In this non-limiting example, the components 110, 112 are components of a knee brace. The spring assembly 100 has a spring 102 and two cylindrical components 104, 106. The cylindrical component 104 has a connection component 108 to which component 112 is coupled. The cylindrical component 106 is coupled to component 110 via a connection component (not shown). As depicted in FIG. 4 solely for exemplary purposes, the preload created by the spring assembly 100 creates sufficient force to cause component 110 to be suspended above the flat surface on which the assembly 100 rests.

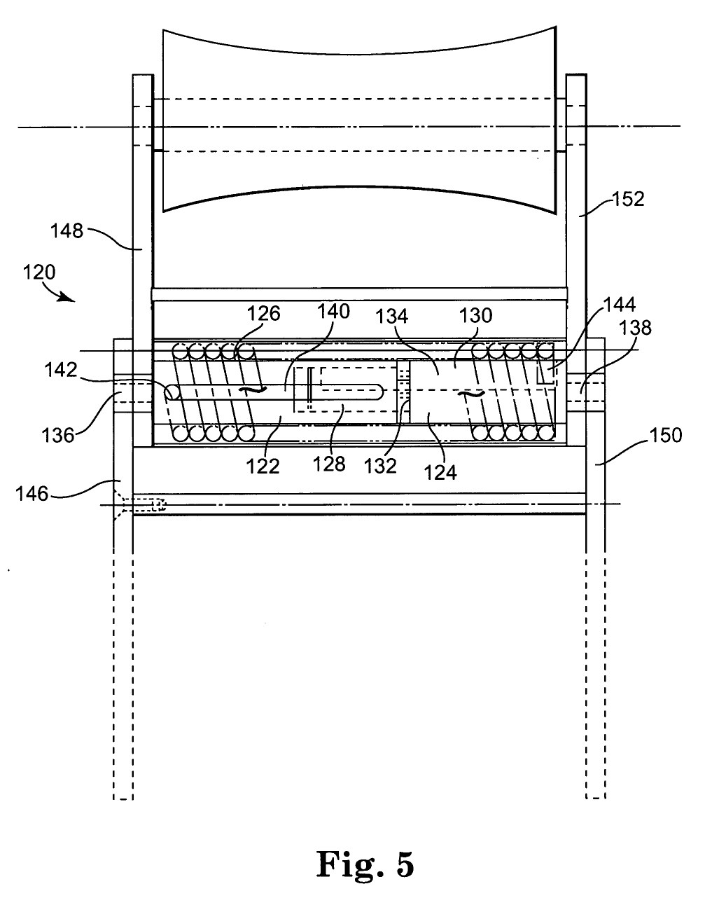

FIG. 5 depicts a schematic representation of a spring assembly 120 that is operably coupled to a knee brace, according to one aspect of the present invention. The spring assembly 120 has a female component 122, a male component 124, and a spring 126. The male component 124 has a first portion (shown schematically with broken lines at 128) that is positioned within the female component 122, a second portion 130, and a connection component 138. The male component 124 also has a spring receiving channel shown schematically with broken lines at 134. The female component 122 has an opening (not shown) in which the first portion 128 has been positioned, a spring receiving channel 140, and connection component 136. The spring 126 encircles portions of the male 124 and female 122 components and has a first end 142 engageably positioned in the spring receiving channel 140 in the female component 122 and a second end depicted schematically with broken lines at 144 engageably positioned in the spring receiving channel 134 in the male component 124. Components 146, 148, 150, 152 of the knee brace are operably coupled at the connection components 136, 138 to the spring assembly 120. According to one embodiment, two opposing components (that is, positioned on opposing connection components and also on opposite sides of the spring assembly 120) have “D-shaped” apertures that engageably mate with the “D-shaped” connection components 136, 138 while the other two components have circular apertures that allow for insertion of the connection components 136, 138 but do not engageably mate with the components 136, 138 (and thus are not placed under torsion force by the spring assembly 120). For example, according to one embodiment, component 148 and component 150 have engaging “D-shaped” apertures such that each of the components 148, 150 are placed under torsion force by the spring assembly 120 while components 146, 152 have circular apertures such that neither of the components 146, 152 is placed under torsion force by the spring assembly 120. Alternatively, components 146, 152 can have the “D-shaped” apertures and components 148, 150 can have the circular apertures.

In an alternative embodiment of the present invention, FIG. 5 depicts a schematic representation of a spring assembly that is operably coupled to an exercise apparatus. The exercise apparatus, according to one embodiment, is any known exercise device that utilizes or might be able to utilize a spring assembly with a preload. In a further alternative, spring assemblies of the present invention can also be used with any other device or component known to require or be able to utilize a torsional spring.

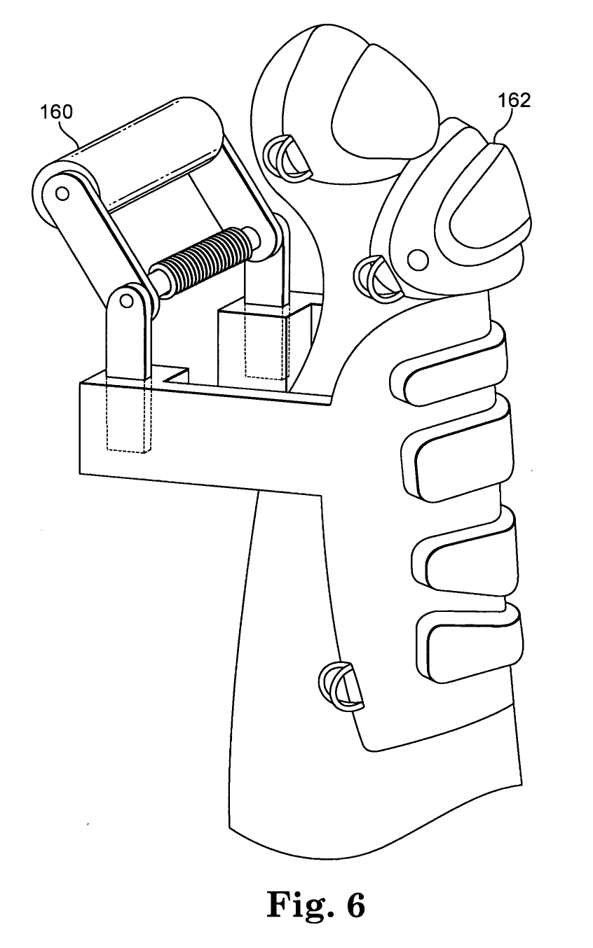

In a further embodiment, FIG. 6 depicts a schematic representation of a spring assembly 160 of the present invention that is operably coupled to a limb protection apparatus 162. The limb protection apparatus 162, according to one embodiment, is a leg guard or shin guard such as, for example, a leg guard warn by a baseball catcher or a shin guard worn by a soccer player. Alternatively, the apparatus 162 is any known limb protection apparatus that utilizes or might be able to utilize a spring assembly with a preload.

According to one embodiment, the combination of the spring assembly 160 with a limb protection apparatus 162 in FIG. 6 provides not only limb protection but also some benefits of a leg brace as well. That is, the combination results in both protection and support or bracing benefits for the limb.

Although the present invention has been described with reference to preferred embodiments, persons skilled in the art will recognize that changes may be made in form and detail without departing from the spirit and scope of the invention.

What is claimed is:

1. A spring assembly comprising:
(a) a first substantially cylindrical component;
(b) a second substantially cylindrical component coupled with the first substantially cylindrical component;
(c) a coupling component associated with the first and second substantially cylindrical components, the coupling component configured to prevent full 360 degree rotation of the first and second substantially cylindrical components in relation to each other; and
(d) a spring encircling at least a portion of the first and second components, the spring comprising a first end removably coupled to the first component and a second end removably coupled to the second component,
wherein the spring has a pre-load tension created by the first and second components, and
further wherein the spring assembly is operably coupled to a limb protection apparatus.

2. The spring assembly of claim 1, wherein the first substantially cylindrical component comprises a first end defining an opening and the second substantially cylindrical component comprises a first portion configured to be rotatably positioned within the opening of the first substantially cylindrical component.

3. The spring assembly of claim 1, wherein the coupling component comprises a first protrusion extending axially from the first end of the first substantially cylindrical component, the first protrusion configured to operably engage with a second protrusion on the second substantially cylindrical component to prevent full rotation of either of the first or second substantially cylindrical components in relation to the other.

4. The spring assembly of claim 3, wherein the first substantially cylindrical component comprises a first end defining an opening and the second substantially cylindrical component comprises a first portion and a second portion, the first portion configured to be rotatably positioned within the opening of the first substantially cylindrical component.

5. The spring assembly of claim 4, wherein the first portion has a smaller diameter than the second portion.

6. The spring assembly of claim 5, wherein the smaller diameter of the first portion creates a shoulder on the second substantially cylindrical component.

7. The spring assembly of claim 6, wherein the second protrusion extends axially from the shoulder of the second substantially cylindrical component.

8. The spring assembly of claim 6, wherein the second protrusion extends radially from the first portion of the second substantially cylindrical component.

9. The spring assembly of claim 1, wherein the first end of the spring is removably coupled to the first substantially cylindrical component at a first spring receiving aperture and wherein the second end of the spring is removably coupled to the second substantially cylindrical component at a second spring receiving aperture.

10. The spring assembly of claim 9, wherein the first and second spring receiving apertures are channel-shaped.

11. The spring assembly of claim 1, wherein the first substantially cylindrical component further comprises a first connection component at an end opposite the coupling component and the second substantially cylindrical component comprises a second connection component at an end opposite the coupling component.

12. The spring assembly of claim 11, wherein the first and second connection components are axial protrusions, wherein each of the first and second connection components are connected with the limb protection apparatus.

13. The spring assembly of claim 12, wherein each of the first and second connection components has a circular shape having a flat surface along its circumference.

14. A limb protection apparatus comprising:
(a) a spring assembly comprising:
(i) a first substantially cylindrical component;
(ii) a second substantially cylindrical component coupled with the first substantially cylindrical component;
(iii) a coupling component associated with a first end of the first and second substantially cylindrical components, the coupling component configured to prevent full 360 degree rotation of the first and second substantially cylindrical components in relation to each other;
(iv) a spring encircling at least a portion of the first and second components, the spring comprising a first end removably coupled to the first component and a second end removably coupled to the second component;
(v) a first connection component associated with a second end of the first substantially cylindrical component; and
(vi) a second connection component associated with a second end of the second substantially cylindrical component,
wherein the spring has a pre-load tension created by the first and second components; and
(b) a limb guard component operably coupled with the first and second connection components.

15. A limb protection apparatus comprising:
(a) a torsion spring assembly having a preload, the spring assembly comprising:
(i) a first substantially cylindrical component comprising a first connection component and a first coupling component;
(ii) a second substantially cylindrical component comprising a second connection component and a second coupling component, the second coupling component rotatably coupled with the first coupling component, whereby the first substantially cylindrical component rotates less than 360 degrees in relation to the second substantially cylindrical component; and
(iii) a spring disposed substantially around the first and second substantially cylindrical components, the spring connected at a first end to the first substantially cylindrical component and at a second end to the second substantially cylindrical component; and
(b) a first member operably coupled to the first connection component; and
(c) a second member operably coupled to the second connection component.
(d) a limb guard component operably coupled to the first and second members.
